(19) United States
(12) Patent Application Publication
(10) Pub. No.: US 2016/0175559 A1
(43) Pub. Date: Jun. 23, 2016

(54) Medical Devices for Delivery and Retention of Bioactive Agents

(71) Applicant: COOK MEDICAL TECHNOLOGIES LLC, BLOOMINGTON, IN (US)

(72) Inventors: Colleen Gemborys, Bloomington, IN (US); Steve Pendleton, Spencer, IN (US); Tyler Bunch, Bloomington, IN (US)

(21) Appl. No.: 14/955,340

(22) Filed: Dec. 1, 2015

Related U.S. Application Data
(60) Provisional application No. 62/096,201, filed on Dec. 23, 2014.

(51) Int. Cl. A61M 25/00; A61M 25/10

(52) U.S. Cl. CPC A61M 25/007; A61M 25/10; A61M 2025/0073; A61M 2025/1052; A61M 2025/1061

(57) ABSTRACT

Medical devices for local bioactive delivery are described. A medical device has an elongate shaft extending between proximal and distal ends and a distal portion that defines a pigtail. A balloon is disposed on the elongate shaft proximal to the pigtail. The elongate shaft defines a first working lumen with a distal opening disposed between the balloon and the pigtail, and a second working lumen with a distal opening disposed distal to the first working lumen distal opening. Methods of using medical devices are also described.

### FIELD

The disclosure relates generally to the field of medical devices. More particularly, the disclosure relates to the field of medical devices suitable for use in delivery of a bioactive agent to a point of treatment in the body of an animal, such as a human being, methods of using such medical devices, and methods of treating a patient.

### BACKGROUND

Cancer is a leading cause of death worldwide. Current treatments for many cancers include systemic administration of bioactive agents that have negative side effects and that can have significant impact on a patient's quality of life. Local delivery of bioactive agents directly to a point of treatment may provide a route of administration that avoids the side effects and quality of life implications associated with systemic delivery of cancer treatment agents and other bioactive agents. The art does not provide, however, suitable medical devices for achieving suitable local dosages while avoiding systemic spread of the bioactive agent.

Local delivery to points of treatment within vessels, ducts, and other body cavities present additional challenges. For example, during intravesical chemotherapy, a chemotherapy solution is infused into the bladder and then retained in the bladder by the patient for a specified period of time. While this treatment can reduce the chance of recurrence by as much as 50%, the art does not provide suitable medical devices for delivering and retaining a bioactive in the bladder. The art also lacks medical devices suitable for localized infusion for other body locations that present greater access challenges, such as the ureters and renal pelvis. As a result, treatment with localized infusion of a bioactive is not as widely used as it could be despite its potential clinical benefit.

A need exists, therefore, for improved medical devices for delivering a bioactive to a point of treatment and retaining the bioactive at the point of treatment for a desired period of time, and for methods of using such medical devices.

### BRIEF DESCRIPTION OF SELECTED EXAMPLES

Several medical devices are described and illustrated herein.

An example medical device comprises an elongate shaft extending between proximal and distal ends and having a distal portion extending proximally from the distal end and defining a pigtail; a balloon disposed on the elongate shaft proximal to the pigtail, the balloon defining an interior chamber; the elongate shaft defining an inflation lumen extending between an inflation lumen proximal opening and an inflation lumen distal opening disposed within the interior chamber of the balloon; the elongate shaft defining a first working lumen extending between a first working lumen proximal opening and a first working lumen distal opening disposed between the balloon and the pigtail; and the elongate shaft defining a second working lumen extending between a second working lumen proximal opening and a second working lumen distal opening disposed distal to the first working lumen distal opening.

Another example medical device comprises an elongate shaft extending between proximal and distal ends and having a distal portion extending proximally from the distal end and defining a pigtail; a first balloon disposed on the elongate shaft proximal to the pigtail, the balloon defining a first interior chamber; a second balloon disposed on the elongate shaft proximal to the first balloon, the second balloon defining a second interior chamber; the elongate shaft defining a first inflation lumen extending between a first inflation lumen proximal opening and a first inflation lumen distal opening disposed within the interior chamber of the first balloon; the elongate shaft defining a second inflation lumen extending between a second inflation lumen proximal opening and a second inflation lumen distal opening disposed within the interior chamber of the second balloon; the elongate shaft defining a first working lumen extending between a first working lumen proximal opening and a first working lumen distal opening disposed between the first balloon and the distal end; and the elongate shaft defining a second working lumen extending between a second working lumen proximal opening and a second working lumen distal opening disposed between the first and second balloons.

Another example medical device comprises an outer tubular member defining a lumen and extending between an outer tubular member proximal end and an outer tubular member distal end; an elongate shaft extending between an elongate shaft proximal end and an elongate shaft distal end and having a distal portion extending proximally from the elongate shaft distal end and defining a pigtail, the elongate shaft disposed through the lumen of the outer tubular member; the elongate shaft defining a first working lumen extending between a first working lumen proximal opening and a first working lumen distal opening disposed on the pigtail; a balloon disposed on the outer tubular member, the balloon defining an interior chamber; the outer tubular member defining an inflation lumen extending between an inflation lumen proximal opening and an inflation lumen distal opening disposed within the interior chamber of the balloon; and the outer tubular member defining a second working lumen extending between a second working lumen proximal opening and a second working lumen distal opening disposed between the balloon and the outer tubular member distal end.

Several methods of using a medical device are described and illustrated herein.

An example method of using a medical device comprises inserting the distal end of a medical device according to an embodiment into a lumen defined by a tubular structure; inflating the balloon of the medical device so that the exterior surface of the balloon contacts the interior surface of the tubular structure and substantially seals the lumen from fluid flow past the balloon; passing a fluid through a working lumen of the medical device such that the fluid exits an opening of the working lumen distal to the balloon and enters the lumen of the tubular structure; retaining the fluid in the lumen of the tubular structure for a period of time; deflating the balloon of the medical device; and withdrawing the medical device from the lumen of the tubular structure.

Additional understanding of the claimed medical devices and methods can be obtained by reviewing the description of selected examples, below, with reference to the appended drawings.

### BRIEF DESCRIPTION OF THE DRAWINGS

FIG. 1 is a perspective view, partially broken away, of a first example medical device.

FIG. 2A is an enlarged sectional view, partially broken away, of the medical device illustrated in FIG. 1, taken along line 2A-2A.

FIG. 2B is an enlarged sectional view, partially broken away, of the medical device illustrated in FIG. 1, taken along line 2B-2B.

FIG. 2C is an enlarged sectional view, partially broken away, of the medical device illustrated in FIG. 1, taken along line 2C-2C.

FIG. 3 is partial sectional view of a mammalian urinary tract. The medical device illustrated in FIG. 1 is disposed in the urinary tract.

FIG. 4 is a perspective view, partially broken away, of a second example medical device.

FIG. 5 is a perspective view of an alternative distal portion for the example medical device illustrated in FIG. 4.

FIG. 6 is a perspective view, partially broken away, of a third example medical device

FIG. 7A is an enlarged sectional view, partially broken away, of the medical device illustrated in FIG. 6, taken along line 7A-7A.

FIG. 7B is an enlarged sectional view, partially broken away, of the medical device illustrated in FIG. 6, taken along line 7B-7B.

FIG. 7C is an enlarged sectional view, partially broken away, of the medical device illustrated in FIG. 6, taken along line 7C-7C.

FIG. 7D is an enlarged sectional view, partially broken away, of the medical device illustrated in FIG. 6, taken along line 7D-7D.

FIG. 7E is an enlarged sectional view, partially broken away, of the medical device illustrated in FIG. 6, taken along line 7E-7E.

FIG. 7F is an enlarged sectional view, partially broken away, of the medical device illustrated in FIG. 6, taken along line 7F-7F.

FIG. 8 is a perspective view, partially broken away, of a fourth example medical device.

FIG. 9 is a perspective view, partially broken away, of a fifth example medical device.

FIG. 10 is a flowchart representation of an example method of using a medical device.

### DETAILED DESCRIPTION OF SELECTED EXAMPLES

The following detailed description and appended drawings describe and illustrate various examples contemplated by the inventors. The description and drawings serve to enable one skilled in the art to make and use the inventive medical devices and methods; they are not intended to limit the scope of the invention or the protection sought in any manner. The invention is capable of being practiced or carried out in various ways; the examples described herein are merely examples of these various ways and are not exhaustive. As such, the language used in the description is to be given the broadest possible scope and meaning.

Unless otherwise defined herein, scientific and technical terms used in connection with the invention shall have the meanings that are commonly understood by those of ordinary skill in the art. Further, unless otherwise required by context, singular terms shall include pluralities and plural terms shall include the singular.

As used herein, the term ‘pigtail’ refers to a loop structure formed in a body. The loop structure can be a partial loop, in which the body defines a curve but does not cross itself, or a loop, in which the body defines a curve and crosses itself. Furthermore, a pigtail that comprises a loop can define a coil. In these structures, the body defines a sequence of loops.

As used herein, the term ‘bioactive’ and grammatically related terms refer to a substance that has a biological effect in an animal.

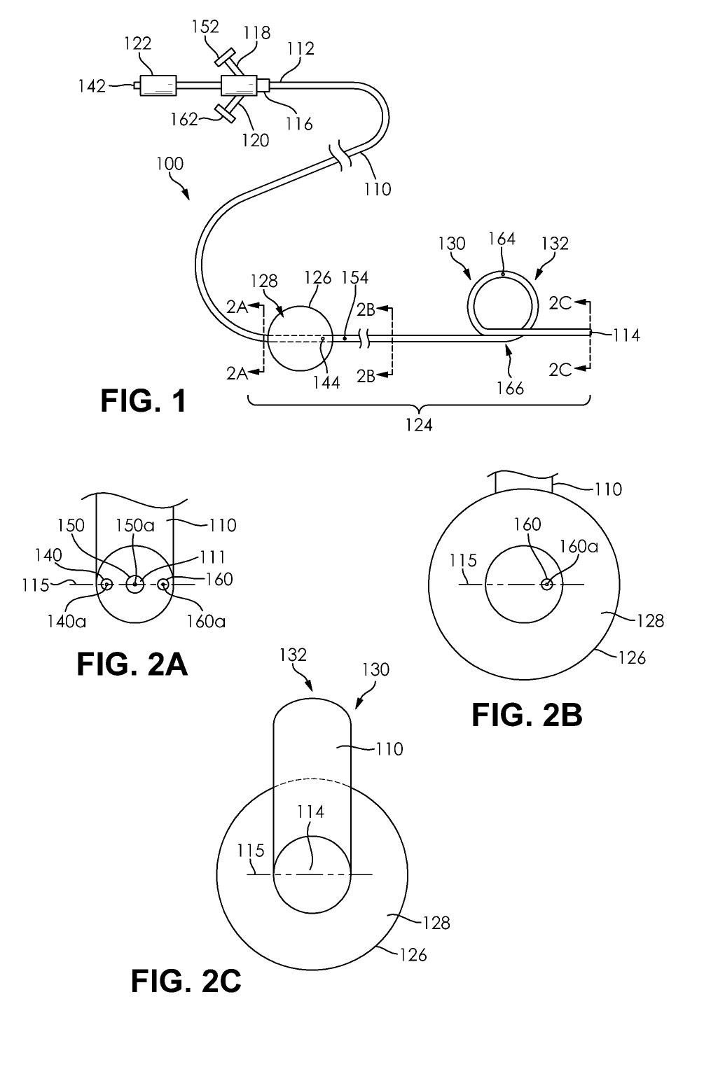

FIGS. 1, 2A, 2B and 2C illustrate a first example medical device 100. As described in detail below, the medical device 100 can be used to deliver a bioactive to a point of treatment and to retain the bioactive at the point of treatment for a desired period of time. If desired, the medical device 100 can be used to remove a delivered bioactive from a point of treatment.

The medical device 100 includes an elongate shaft 110 extending between a proximal end 112 and a distal end 114. A hub 116 is disposed on the proximal end 112 and includes first 118 and second 120 sidearm connectors and in-line connector 122.

A distal portion 124 of the elongate shaft 110 extends from the distal end 114 and toward the proximal end 112. While the distal portion of a medical device according to a particular embodiment can have any suitable axial length relative to the overall axial length of the medical device, the distal portion 124 of medical device 100 has an axial length that is about 10% of the overall axial length of medical device 100.

A balloon 126 is attached to distal portion 124 of the elongate shaft 110. The balloon 126 defines an interior chamber 128 and has uninflated and inflated configurations. FIG. 1 illustrates the balloon 126 in the inflated configuration.

Any suitable balloon can be used in a medical device according to a particular embodiment. Indeed, the balloon used in a particular medical device can have any suitable composition, size, shape and configuration. A skilled artisan will be able to select a suitable balloon for use in a medical device according to a particular embodiment based on various considerations, including the point or points of treatment within the body at which the medical device is intended to be used. In the illustrated example, balloon 126 is a spherical balloon. Elongate balloons and balloons having others shapes and configurations can also be used. Balloons having additional elements, such as cutting surfaces or the like, can be also be used.

The distal portion 124 of the elongate shaft 110 defines a pigtail 130. Any suitable pigtail can be used in a medical device according to a particular embodiment. Indeed, the pigtail used in a particular medical device can have any suitable loop structure, including a partial loop, a loop, and a coil. A skilled artisan will be able to select a suitable loop structure for the pigtail in a medical device according to a particular embodiment based on various considerations, including the point or points of treatment within the body at which the medical device is intended to be used. In the illustrated example, pigtail 130 comprises a loop 132, but only a single loop. The pigtail used in a particular medical device can also be positioned at any suitable axial location on the distal portion of the elongate shaft of the medical device. In the illustrated example, pigtail 130 is positioned near the distal end 114 of the elongate shaft 110, but proximally from the distal end 114 such that the distal end 114 is not positioned within the loop 132 of the pigtail 130. Other examples of suitable axial locations for the pigtail include axial locations within the distal portion that place the distal end of the elongate shaft within the loop of the pigtail.

The elongate shaft 110 defines inflation lumen 140 that extends between inflation lumen proximal opening 142, defined by in-line connector 122, and inflation lumen distal opening 144, defined by the elongate shaft 110. As best illustrated in FIG. 1, inflation lumen distal opening 144 is positioned within interior chamber 128 of balloon 126. As such, passage of a suitable inflation fluid, such as saline, through inflation lumen 140 and into the interior chamber 128 of balloon 126 moves the balloon 126 from its uninflated configuration to its inflated configuration. Similarly, withdrawal of inflation fluid from the interior chamber 128 into the inflation lumen 140 moves the balloon 126 from its inflated configuration to its uninflated configuration. A syringe or other suitable storage device containing a suitable volume of a suitable inflation fluid can be attached to in-line connector 122 in the conventional manner to permit a user to operate the balloon 126 in this manner.

The elongate shaft 110 defines first working lumen 150 that extends between first working lumen proximal opening 152, defined by first sidearm connector 118, and first working lumen distal opening 154, defined by elongate shaft 110. As best illustrated in FIG. 1, first working lumen distal opening 154 is positioned axially between balloon 126 and pigtail 130. That is, first working lumen distal opening 154 is positioned on elongate shaft 110 distal to balloon 126 and proximal to pigtail 130. This positioning allows use of first working lumen 150 to deliver a bioactive to a point of treatment in a manner that allows the bioactive to be retained at the point of treatment for a desired period of time, as described in more detail below.

The first working lumen distal opening in a medical device according to a particular embodiment can be axially positioned on the elongate shaft of the medical device at any suitable location between the balloon and pigtail, and a skilled artisan will be able to select an appropriate axial location on the elongate shaft based on various considerations, including the point or points of treatment within the body at which the medical device is intended to be used, the size, shape and/or configuration of the balloon of the medical device, and the size, shape and/or configuration of the pigtail of the medical device. In the illustrated example, first working lumen distal opening 154 is positioned closer to the balloon 126 than to the pigtail 130. This positioning is considered advantageous at least because it is believed to facilitate delivery of a bioactive at points of contact between a body lumen at a point of treatment and the balloon 126 during use of the medical device 100.

The first working lumen 150 can be used to deliver a fluid, such as a treatment fluid containing a bioactive, a wash fluid, or other suitable fluid, to a point of treatment in a body vessel. Introduction of the fluid into first working lumen proximal opening 152, through first working lumen 150 and out of first working lumen distal opening 154 delivers the fluid to the external environment adjacent the first working lumen distal opening 154, such as a point of treatment in a body, as described in detail below. Similarly, by placing suction force on the first working lumen proximal opening 152, a fluid in the external environment adjacent the first working lumen distal opening 154 can be withdrawn from the external environment and into the first working lumen 150, essentially removing the fluid from the external environment adjacent the first working lumen distal opening 154. A syringe or other suitable storage device containing a suitable volume of a suitable fluid can be attached to first sidearm connector 118 in the conventional manner to permit a user to use the first working lumen 150 in this manner.

The elongate shaft 110 defines second working lumen 160 that extends between proximal second working lumen opening 162, defined by second sidearm connector 120, and distal second working lumen opening 164, defined by elongate shaft 110. As best illustrated in FIG. 1, second working lumen distal opening 164 is positioned distal to first working lumen distal opening 154. That is, second working lumen distal opening 164 is positioned on elongate shaft 110 distal to balloon 126 and distal to the first working lumen distal opening 154. This positioning allows use of second working lumen 160 to deliver a bioactive to a point of treatment in a manner that allows the bioactive to be retained at the point of treatment for a desired period of time, as described in more detail below.

The second working lumen distal opening in a medical device according to a particular embodiment can be axially positioned on the elongate shaft of the medical device at any suitable location between the first working lumen distal opening and the distal end of the medical device, and a skilled artisan will be able to select an appropriate axial location on the elongate shaft based on various considerations, including the point or points of treatment within the body at which the medical device is intended to be used, the size, shape and/or configuration of the balloon of the medical device, and the size, shape and/or configuration of the pigtail of the medical device. In the illustrated example, as best illustrated in FIG. 1, second working lumen distal opening 164 is positioned on the pigtail 130. More specifically, second working lumen distal opening 164 is positioned on pigtail 130 at the point on the elongate shaft 110 that is directly opposite the crossover point 166 of the elongate shaft 110 in the loop 132 of the pigtail 130. This positioning is considered advantageous at least because it is believed to facilitate delivery of a bioactive to the distal most locations of a particular point of treatment during use of the medical device 100. Other examples of suitable axial positions for the second working lumen distal opening on the elongate shaft 110 include a position proximal to the pigtail 130, a position distal to the first working lumen distal opening 154 and distal to the pigtail 130, a position distal to the first working lumen distal opening 154 and proximal to the pigtail 130, any suitable position distal to the first working lumen distal opening 154 and on the pigtail 130, and a position distal to the first working lumen distal opening 154 and directly on the distal end 114 of the elongate shaft 110.

Similar to the first working lumen 150, the second working lumen 160 can be used to deliver a fluid, such as a treatment fluid containing a bioactive, a wash fluid, or other suitable fluid, to a point of treatment in a body vessel. Introduction of the fluid into second working lumen proximal opening 162, through second working lumen 160 and out of second working lumen distal opening 164 delivers the fluid to the external environment adjacent the second working lumen distal opening 164, such as a point of treatment in a body, as described in detail below. Similarly, by placing suction force on the second working lumen proximal opening 162, a fluid in the external environment adjacent the second working lumen distal opening 164 can be withdrawn from the external environment and into the second working lumen 160, essentially removing the fluid from the external environment adjacent the second working lumen distal opening 164. A syringe or other suitable storage device containing a suitable volume of a suitable fluid can be attached to second sidearm connector 120 in the conventional manner to permit a user to use the second working lumen 160 in this manner.

Each of the inflation lumen, first working lumen, and second working lumen in a medical device according to a particular embodiment can have any suitable size, shape and configuration. A skilled artisan will be able to select a size, shape and configuration for each of the inflation lumen, first working lumen and second working lumen in a medical device according to a particular embodiment based on various considerations, including, for the inflation lumen, the composition, size, shape and configuration of the balloon and, for the first and second working lumens, the size, shape and configuration of the inflation lumen, the nature of the fluid intended to be passed through the first and/or second working lumens, and any desired relative delivery considerations (e.g., it may be desirable to deliver a greater volume of fluid through the second working lumen than the first working lumen; a second working lumen with a greater cross-sectional area than that of the first working lumen may facilitate achievement of this). For all lumens, any desired overall flexibility for the elongate shaft in a medical device according to a particular embodiment may also be taken into consideration when selecting suitable lumen shapes, sizes and configurations. As best illustrated in FIG. 2A, each of inflation lumen 140, first working lumen 150, and second working lumen 160 in the first example medical device has a circular cross-sectional shape. Furthermore, the lumens 140, 150, 160 have substantially similar cross-sectional areas. Examples of other suitable cross-sectional shapes that can be used for the lumens include kidney-shaped, square, triangular, and other shapes. A combination of different cross-sectional shapes can also be used. Also, it is noted that the lumens can be positioned relative to each other in any suitable manner in a medical device according to a particular embodiment. In the illustrated example, as best illustrated in FIG. 2A, the lumens 140, 150, 160 are positioned side-by-side-by-side such that the central axes 140a, 150a, 160a of lumens 140, 150, 160 lie on a common transverse axis 115 that lies on a plane that orthogonally intersects a plane containing the longitudinal axis 111 of the elongate shaft 110.

The elongate shaft 110 is a solid member that defines the inflation lumen 140, the first working lumen 150, and the second working lumen 160. The elongate shaft 110 can be formed of any suitable material, including known materials used in the manufacture of conventional catheters.

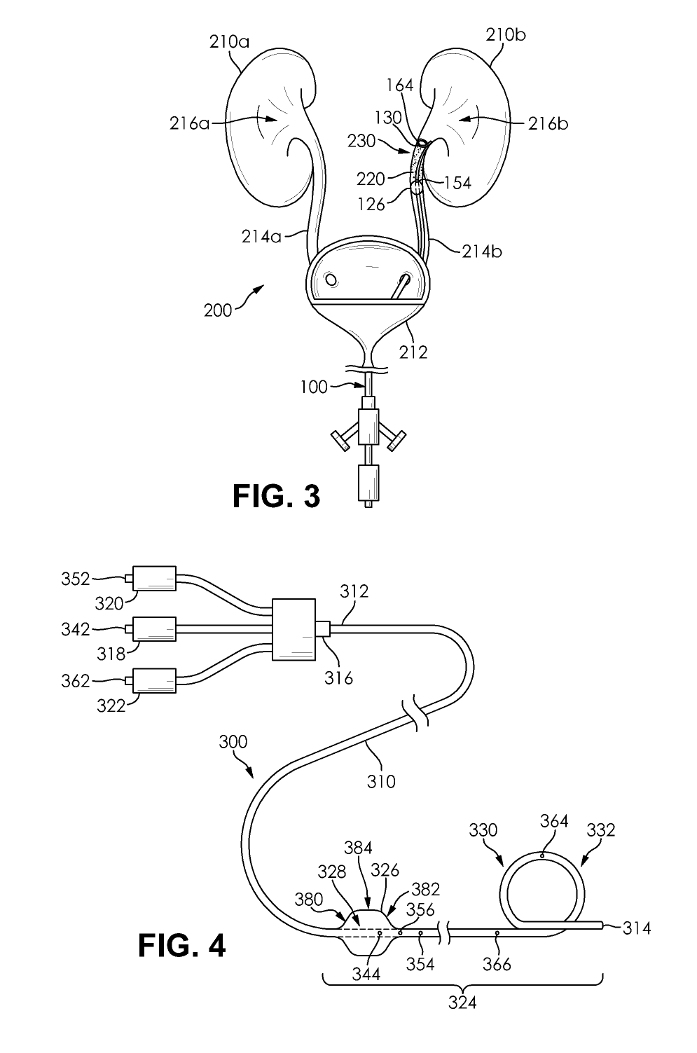

FIG. 3 illustrates a mammalian urinary tract 200, including kidneys 210a, 210b, bladder 212, and ureters 214a, 214b. The medical device 100 is partially disposed in the urinary tract 200 in a manner that allows a user to use the medical device 100 to deliver a fluid 220 containing a bioactive to a point of treatment 230 contained entirely within the ureter 214b by expelling the fluid 220 through one or both of the first working lumen distal opening 154 and the second working lumen distal opening 164. The balloon 126 is in its inflated configuration within the ureter 214b, effectively isolating the point of treatment 230 from the portions of the urinary tract 200 proximal to the balloon 126. As long as balloon 126 is maintained in its inflated configuration, the fluid 220 containing the bioactive can be retained at the point of treatment 230 for a desired period of time, effectively exposing the inner wall of the ureter 214b to the bioactive for the desired period of time. The medical device 100 is positioned such that the pigtail 130 is disposed in the renal pelvis 216b of kidney 210b. Renal pelvis 216a of kidney 210a, and ureter 214a, are free of the medical device 100 in FIG. 3. If desired, the medical device 100 can be used to remove the bioactive from the point of treatment 220 in the ureter 214b by withdrawing the fluid 220 containing the bioactive from the point of treatment 230 using one or both of the first and second working lumens (not illustrated in FIG. 3), as described above.

FIG. 4 illustrates a second example medical device 300. The medical device 300 is similar to the medical device 100 illustrated in FIGS. 1, 2A, 2B, and 2C, and described above, except as detailed below. Thus, the medical device 300 includes an elongate shaft 310 extending between a proximal end 312 and a distal end 314. A hub 316 is disposed on the proximal end 312. A balloon 326 and a pigtail 330 are disposed on a distal portion 324 of the elongate shaft 310.

In this embodiment, hub 316 includes first 318, second 320, and third 322 in-line connectors. The inflation lumen (not visible in FIG. 4) extends between an inflation lumen proximal opening 342 defined by the first 318 in-line connector 318 to an inflation lumen distal opening 344 defined by the elongate shaft 110 and disposed within the interior chamber 328 of the balloon 326.

The first working lumen (not visible in FIG. 4) extends between a first working lumen proximal opening 352 defined by the second in-line connector 320 to a first working lumen distal opening 354 defined by the elongate shaft 310 and disposed between the balloon 326 and the pigtail 330. In this embodiment, the elongate shaft 310 defines a first working lumen intermediate opening 356 that is disposed between the balloon 326 and the first working lumen distal opening 354. The first working lumen intermediate opening 356 provides access to the first working lumen just as the first working lumen distal opening 354 does. Indeed, in the illustrated embodiment, the first working lumen distal opening 354 and the first working lumen intermediate opening 356 have the same size, shape and configuration. It is noted, though, that any suitable size, shape and configuration can be used for the first working lumen intermediate opening 356, including a size, shape and/or configuration that is different from that of the first working lumen distal opening 354.

The second working lumen (not visible in FIG. 4) extends between a second working lumen proximal opening 362 defined by the third in-line connector 322 to a second working lumen distal opening 364 defined by the elongate shaft 310 and disposed on the pigtail 330. In this embodiment, the elongate shaft 310 defines a second working lumen intermediate opening 366 that is disposed between the first working lumen distal opening 354 and the second working lumen distal opening 364. The second working lumen intermediate opening 366 provides access to the second working lumen just as the second working lumen distal opening 364 does. Indeed, in the illustrated embodiment, the second working lumen distal opening 364 and the second working lumen intermediate opening 366 have the same size, shape and configuration. It is noted, though, that any suitable size, shape and configuration can be used for the second working lumen intermediate opening 366, including a size, shape and/or configuration that is different from that of the second working lumen distal opening 364.

The second working lumen distal opening 364 and the second working lumen intermediate opening 366 can be axially positioned at any suitable locations on the elongate shaft 310. In the illustrated embodiment, the second working lumen distal opening 364 is positioned on the pigtail 330 and the second working lumen intermediate opening 366 is positioned proximal to the pigtail 330, but still distal to the first working lumen distal opening 354. This arrangement is considered advantageous at least because it is expected to provide desirable delivery and withdrawal capabilities for the medical device 300 when the pigtail 330 is disposed in particular anatomies, such as the renal pelvis.

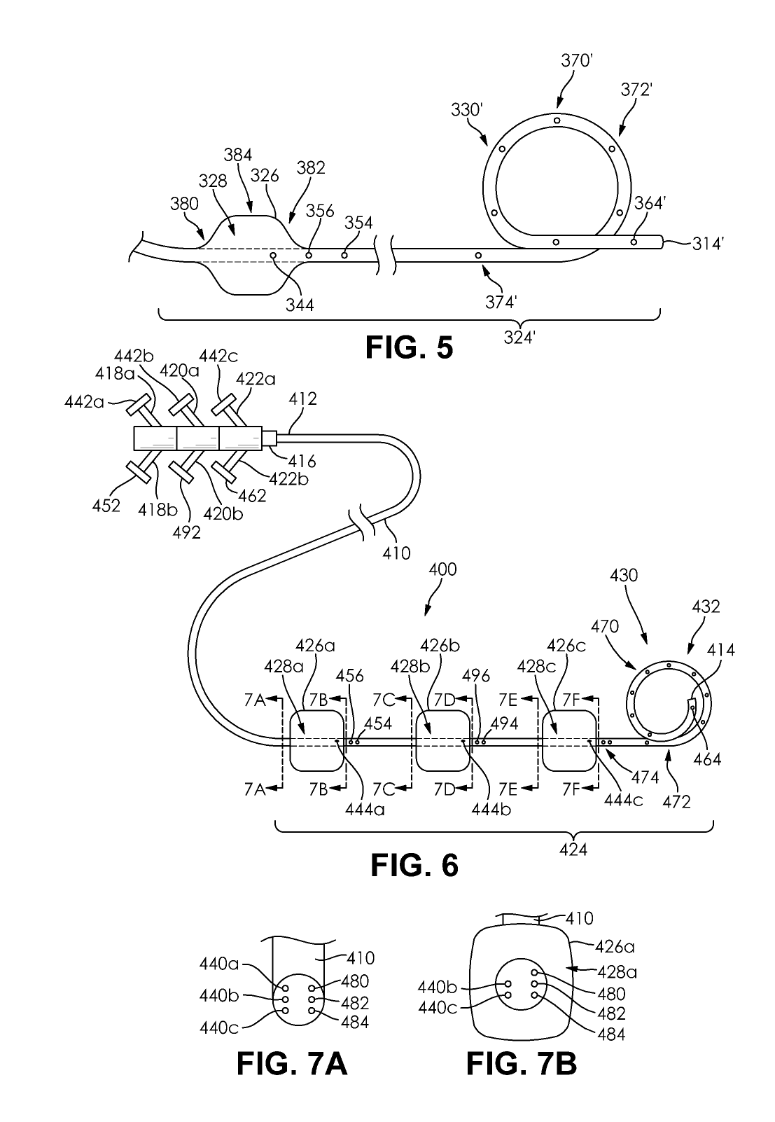

FIG. 5 illustrates an alternative distal portion 324′ for the example medical device 300. In this alternative example, the second working lumen extends between a second working lumen proximal opening (not illustrated in FIG. 5) to a second working lumen distal opening 364′ defined by the elongate shaft 310′ and disposed on the elongate shaft 310′ distal to the pigtail 330′. In this embodiment, the elongate shaft 310′ defines a series of second working lumen intermediate openings 370′ that is disposed between the first working lumen distal opening 354 and the second working lumen distal opening 364′. Each of the series of second working lumen intermediate opening 370′ provides access to the second working lumen just as the second working lumen distal opening 364′ does. Indeed, in the illustrated embodiment, the second working lumen distal opening 364′ and each of the series of second working lumen intermediate openings 370′ have the same size, shape and configuration. It is noted, though, that any suitable size, shape and configuration can be used for each of the series of second working lumen intermediate openings 370′, including a size, shape and/or configuration that is different from that of the second working lumen distal opening 364′.

If included, the series of second working lumen intermediate openings 370′ can be arranged on the elongate shaft 310′ in any suitable manner. For example, as illustrated in FIG. 5, a first group 372′ of the second working lumen intermediate openings 370′ can be disposed on the pigtail 330′ and a second group 374′ of the series of second working lumen intermediate openings 370′ can be disposed on the elongate shaft 310′ proximal to the pigtail 330′. It is considered advantageous that two or more of the series of second working lumen intermediate openings 370′ be disposed on the pigtail 330′ and at least one of the series of second working lumen intermediate openings 370′ be disposed on the elongate shaft 310′ proximal to the pigtail 330′.

In the embodiment illustrated in FIG. 4, balloon 326 is an elongate balloon with a proximal neck portion 380, a distal neck portion 382, and an intermediate portion 384 between the proximal 380 and distal 382 neck portions. The intermediate portion 384 has a constant outer diameter along its length.

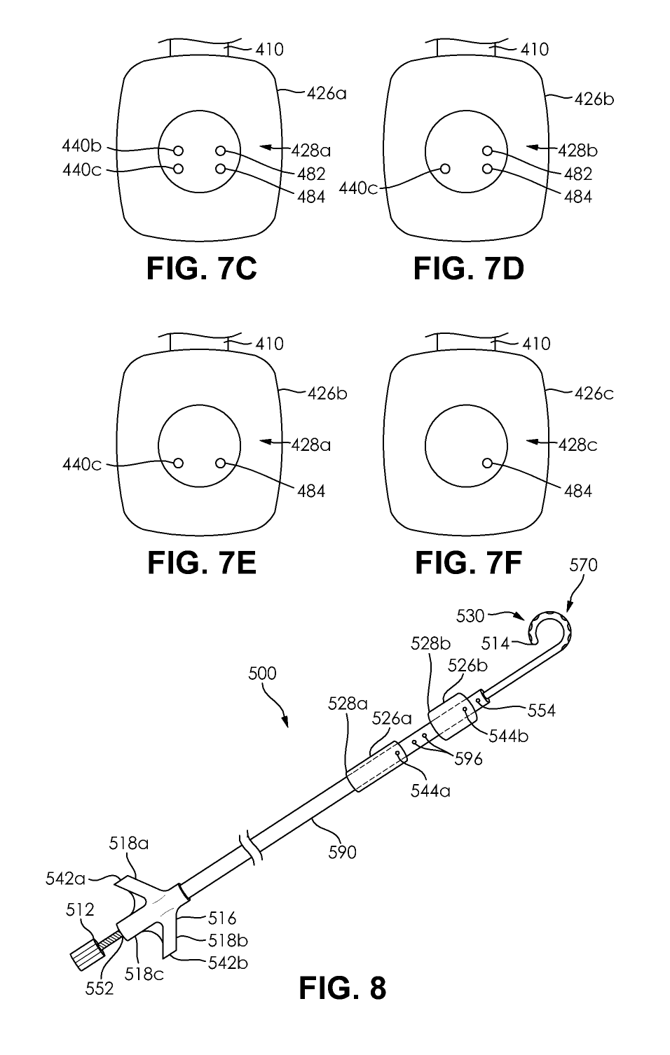

FIGS. 6, 7A, 7B, 7C, 7D, 7E, and 7F illustrate a third example medical device 400. The medical device 400 is similar to the medical device 100 illustrated in FIGS. 1, 2A, 2B, and 2C, and described above, except as detailed below. Thus, the medical device 400 includes an elongate shaft 410 extending between a proximal end 412 and a distal end 414. A hub 416 is disposed on the proximal end 412. A pigtail 430 is disposed on a distal portion 424 of the elongate shaft 410.

In this embodiment, hub 416 includes a first pair 418a, 418b of sidearm connectors, a second pair 420a, 420b of sidearm connectors, and a third pair 422a, 422b of sidearm connectors. Also, the medical device includes first 426a, second 426b, and third 426c balloons attached to the elongate shaft 410. The first balloon 426a defines a first interior chamber 428a. The second balloon 426b defines a second interior chamber 428b. The third balloon 426c defines a third interior chamber 428c. In the illustrated embodiment, each of the balloons 426a, 426b, 426c is non-spherical and defines a substantially constant outer diameter along a central portion of its axial length. It is noted that, while the balloons 426a, 426b, 426c in the illustrated embodiment have the same size, shape and configuration, any combination of balloon sizes, shapes and configurations can be used in a medical device according to a particular embodiment. Use of multiple balloons having the same size, shape and configuration is considered advantageous for particular medical devices, such as medical devices intended to be used within a body lumen having a substantially constant inner diameter, such as a ureter within a mammalian urinary tract.

Each of the balloons 426a, 426b, 426c has an uninflated and an inflated configuration and can transition between its configurations independently of the other two balloons of the balloons 426a, 426b, 426c because each of the balloons 426a, 426b, 426c has its own inflation lumen. Thus, a first inflation lumen 440a extends between a first inflation lumen proximal opening 442a defined by a sidearm connector 418a of the first pair of sidearm connectors 418a, 418b to a first inflation lumen distal opening 444a defined by the elongate shaft 410 and disposed within the interior chamber 428a of the first balloon 426a. The first inflation lumen provides inflation control for the first balloon 426a. Similarly, a second inflation lumen 440b extends between a second inflation lumen proximal opening 442b defined by a sidearm connector 420a of the second pair of sidearm connectors 420a, 420b to a second inflation lumen distal opening 444b defined by the elongate shaft 410 and disposed within the interior chamber 428a of the second balloon 426b. The second inflation lumen provides inflation control for the second balloon 426b. Also similarly, a third inflation lumen 440c extends between a third inflation lumen proximal opening 442c defined by a sidearm connector 422a of the third pair of sidearm connectors 422a, 422b to a first inflation lumen distal opening 444c defined by the elongate shaft 410 and disposed within the interior chamber 428c of the third balloon 426c. The third inflation lumen provides inflation control for the third balloon 426b.

A first working lumen 480 extends between a first working lumen proximal opening 452 defined by a sidearm connector 418b of the first pair of sidearm connectors 418a, 418b to a first working lumen distal opening 454 defined by the elongate shaft 410 and disposed between the first balloon 426a and the second balloon 426b. In this embodiment, the elongate shaft 410 defines a first working lumen intermediate opening 456 that is disposed between the first balloon 426a and the first working lumen distal opening 454. The first working lumen intermediate opening 456 provides access to the first working lumen just as the first working lumen distal opening 454 does. Indeed, in the illustrated embodiment, the first working lumen distal opening 454 and the first working lumen intermediate opening 456 have the same size, shape and configuration. It is noted, though, that any suitable size, shape and configuration can be used for the first working lumen intermediate opening 456, including a size, shape and/or configuration that is different from that of the first working lumen distal opening 454.

A second working lumen 482 extends between a second working lumen proximal opening 492 defined by a sidearm connector 420b of the second pair of sidearm connectors 420a, 420b to a second working lumen distal opening 494 defined by the elongate shaft 410 and disposed between the second balloon 426b and the third balloon 426c. In this embodiment, the elongate shaft 410 defines a first working lumen intermediate opening 496 that is disposed between the second balloon 426b and the second working lumen distal opening 494. The second working lumen intermediate opening 496 provides access to the second working lumen just as the second working lumen distal opening 494 does. Indeed, in the illustrated embodiment, the second working lumen distal opening 494 and the second working lumen intermediate opening 496 have the same size, shape and configuration. It is noted, though, that any suitable size, shape and configuration can be used for the second working lumen intermediate opening 496, including a size, shape and/or configuration that is different from that of the second working lumen distal opening 494.

A third working lumen 484 extends between a third working lumen proximal opening 462 defined by a sidearm connector 422b of the third pair of sidearm connectors 422a, 422b to a third working lumen distal opening 464 defined by the elongate shaft 410 and disposed on the elongate shaft 410 on the pigtail 430. In this embodiment, the elongate shaft 410 defines a series of third working lumen intermediate openings 470 that is disposed between the third balloon 426c and the third working lumen distal opening 464. Each opening of the series of third working lumen intermediate openings 470 provides access to the third working lumen just as the third working lumen distal opening 464 does. Indeed, in the illustrated embodiment, the third working lumen distal opening 464 and each opening of the series of third working lumen intermediate openings 470 have the same size, shape and configuration. It is noted, though, that any suitable size, shape and configuration can be used for each opening of the series of third working lumen intermediate openings 470, including a size, shape and/or configuration that is different from that of the third working lumen distal opening 464.

In this embodiment, a first group of openings 472 of the third working lumen intermediate openings 470 is disposed on the pigtail 430 and a second group of openings 474 of the series of third working lumen intermediate openings 470 can be disposed on the elongate shaft 410 proximal to the pigtail 430 and adjacent the third balloon 426c. As noted above, other arrangements of third working lumen intermediate openings 470 on the elongate shaft 410 can be used.

In this embodiment, the distal end 414 of the elongate shaft 410 is positioned within the loop 432 of the pigtail 430.

Medical devices according to some examples include an elongate shaft disposed through an outer catheter. Similar to the examples described above, the elongate shaft in these examples defines a pigtail. The elongate shaft also defines a working lumen and one or more openings through the wall of the elongate shaft to provide access to the working lumen. In contrast to the examples described above, however, the elongate shaft is disposed through an outer tubular member. One or more balloons can be associated with the outer tubular member. Also, the outer tubular member can define one or more inflation lumens, one or more working lumens, and one or more openings through its wall to provide access to its one or more working lumens.

FIG. 8 illustrates an example medical device 500 having this structural arrangement. The medical device 500 includes an elongate shaft 510 extending between a proximal end 512 and a distal end 514. A pigtail 530 is disposed on a distal portion 524 of the elongate shaft 510. The elongate shaft 510 defines a working lumen extending between the proximal end 512 and a group of openings 570 disposed on the pigtail 530.

In this embodiment, the elongate shaft 510 is disposed through an outer tubular member 590. A hub 516 includes a first pair 518a, 518b of sidearm connectors, and a third connector 518c. Also, first 526a and second 526b balloons are disposed on the outer tubular member 590. The first balloon 526a defines a first interior chamber 528a. The second balloon 526b defines a second interior chamber 528b. It is noted that, while the balloons 526a, 526b in the illustrated embodiment have different sizes, any combination of balloon sizes, shapes and configurations can be used in a medical device according to a particular embodiment.

Each of the balloons 526a, 526b has an uninflated and an inflated configuration and can transition between its configurations independently of the other balloon because each of the balloons 526a, 526b has its own inflation lumen. Thus, a first inflation lumen 540a extends between a first inflation lumen proximal opening 542a defined by a sidearm connector 518a to a first inflation lumen distal opening 544a defined by the defined by the outer tubular member 590 and disposed within the interior chamber 528a of the first balloon 526a. The first inflation lumen 540a provides inflation control for the first balloon 526a. Similarly, a second inflation lumen 540b extends between a second inflation lumen proximal opening 542b defined by a sidearm connector 518b to a second inflation lumen distal opening 544b defined by the elongate shaft 590 and disposed within the interior chamber 528a of the second balloon 526b. The second inflation lumen 540b provides inflation control for the second balloon 526b.

A first working lumen 580 extends between a first working lumen proximal opening 552 defined by connector 518c to a working lumen distal opening 554 defined by the outer tubular member 590 and disposed distal to the second balloon 526b. Additional openings can be defined by the outer tubular member 590, such as working lumen intermediate openings 596 disposed between the first 526a and second 526b balloons.

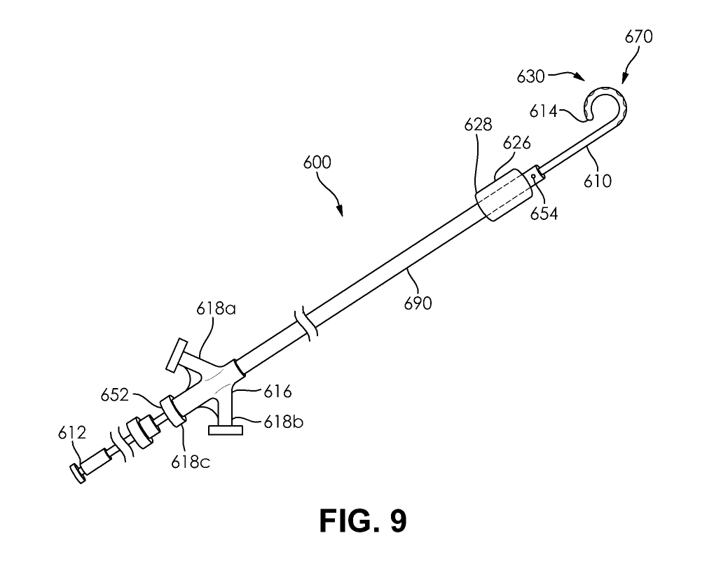

FIG. 9 illustrates another example medical device 600 having the elongate shaft and outer catheter structure. The medical device 600 is similar to the medical device 500 illustrated in FIG. 8 and described above, except as detailed below. Thus, the medical device 600 includes an elongate shaft 610 extending between a proximal end 612 and a distal end 614. A pigtail 630 is disposed on a distal portion 624 of the elongate shaft 610. The elongate shaft 610 defines a working lumen extending between the proximal end 612 and a group of openings 670 disposed on the pigtail 630. The elongate shaft 610 is disposed through an outer tubular member 690. A hub 616 includes a first pair 618a, 618b of sidearm connectors, and a third connector 618c. Also, a single balloon 626 balloon is disposed on the outer tubular member 690. The balloon 626 defines an interior chamber 628a.

The balloon 626 has an uninflated and an inflated configuration and can transition between its configurations. An inflation lumen 640 extends between an inflation lumen proximal opening 642 defined by a sidearm connector 618a to an inflation lumen distal opening 644 defined by the outer tubular member 690 and disposed within the interior chamber 628 of the balloon 626. The inflation lumen 640 provides inflation control for the balloon 626.

A first working lumen 680 extends between a working lumen proximal opening 652 defined by connector 618c to a working lumen distal opening 654 defined by the outer tubular member 690 and disposed distal to the balloon 626.

The medical devices can be used in a variety of ways, in a variety of manners, and in a variety of locations.

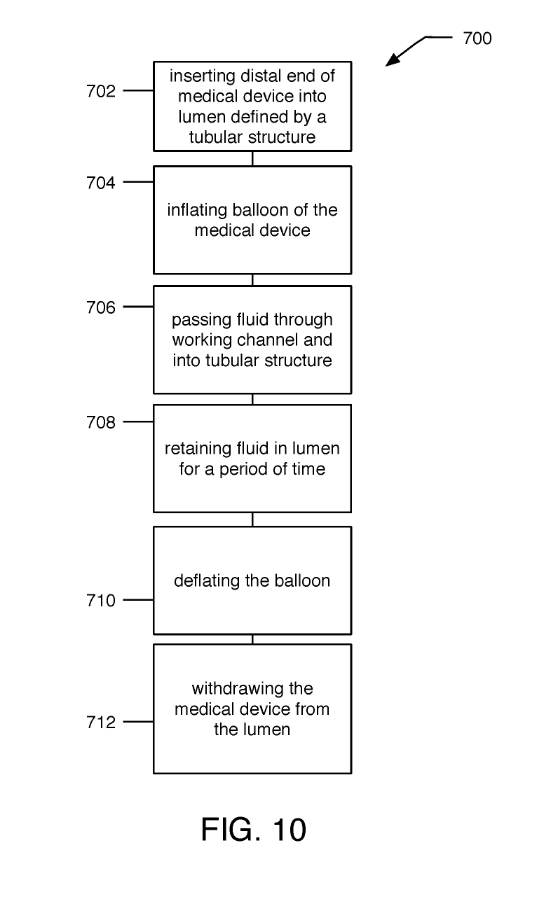

FIG. 10 illustrates a flowchart representation of a first example method 700 of using a medical device. A first step 702 comprises inserting the distal end of a medical device according to an embodiment, such as medical devices 100, 200, 300, 400, 500 and 600, into a lumen defined by a tubular structure. The distal end of the medical device should be advanced into the lumen by a distance sufficient to allow the balloon of the medical device to enter the lumen. Another step 704 comprises inflating the balloon of the medical device so that the exterior surface of the balloon contacts the interior surface of the tubular structure and substantially seals the lumen from fluid flow past the balloon. Another step 706 comprises passing a fluid through a working lumen of the medical device such that the fluid exits an opening of the working lumen distal to the balloon and enters the lumen of the tubular structure. Another step 708 comprises retaining the fluid in the lumen of the tubular structure for a period of time. Another step 710 comprises deflating the balloon of the medical device. Another step 712 comprises withdrawing the medical device from the lumen of the tubular structure.

The inventive medical devices can be used in the treatment of patients, including humans and other animals.

It is noted that, in all methods, individual steps can be performed in any suitable order.

In all methods, the fluid used can comprise one or more bioactives. In these methods, any suitable bioactive, or bioactive, can be used. In a particular method according to a specific example, the specific bioactive, or bioactives, selected will depend upon several considerations, including the desired effect and the type of treatment and/or procedure in which the medical device is intended to be used. Examples of suitable bioactives include anti-cancer agents, such as paclitaxel, tamoxifen citrate, and Taxol® or derivatives thereof; anthracyclines, such as doxorubicin; pyrimidine analogs, such as 5-fluorouracil; nucleoside analogs, such as gemcitabine; platinum-based antineoplastics, such as cisplatin, carboplatin, oxaliplatin, satraplatin, picoplatin, nedaplatin, triplatin, and lipoplatin; immunosuppressive agents, such as cyclosporine and sirolimus; and other anti-cancer chemotherapeutic agents. Monoclonal and polyclonal antibodies can also be used as a bioactive in the methods and medical devices described herein. For example, recombinant humanized angiogenesis inhibiting monoclonal antibodies, such as Bevacizumab, are suitable. Also, chimeric monoclonal antibodies that inhibit epidermal growth factor receptor (EGFR), such as Cetuximab, are suitable. Also, anti-idiotype tumor antigen epitope mirroring monoclonal antibodies, such as Abagovomab, are suitable. Other examples of bioactives that can be used in the methods and medical devices include, but are not limited to, heparin, covalent heparin or another thrombin inhibitor, hirudin, hirulog, argatroban, D-phenylalanyl-L-poly-L-arginyl chloromethyl ketone, or another antithrombogenic agent, or mixtures thereof; urokinase, streptokinase, a tissue plasminogen activator, or another thrombolytic agent, or mixtures thereof; a tyrosine-kinase inhibitor, such as Imatinib; a fibrinolytic agent; a vasospasm inhibitor; a calcium channel blocker, a nitrate, nitric oxide, a nitric oxide promoter or another vasodilator; an antimicrobial agent or antibiotic; aspirin, ticlopidine, a glycoprotein IIb/IIIa inhibitor or another inhibitor of surface glycoprotein receptors, or another antiplatelet agent; colchicine or another antimitotic, or another microtubule inhibitor, dimethylsulfoxide (DMSO), a retinoid or another antisecretory agent; cytochalasin or another actin inhibitor; or a remodeling inhibitor; deoxyribonucleic acid, an antisense nucleotide or another agent for molecular genetic intervention; methotrexate or another antimetabolite or antiproliferative agent; dexamethasone, dexamethasone sodium phosphate, dexamethasone acetate or another dexamethasone derivative, or another anti-inflammatory steroid or non-steroidal anti-inflammatory agent; tripodal (aPDGF antagonist), angiopeptin (a growth hormone antagonist), angiogenin or other growth factors, or an anti-growth factor antibody, or another growth factor antagonist; dopamine, bromocriptine mesylate, pergolide mesylate or another dopamine agonist; 60Co, 192Ir, 32P, 111In, 90Y, 99mTc or another radiotherapeutic agent; iodine-containing compounds, barium-containing compounds, gold, tantalum, platinum, tungsten or another heavy metal functioning as a radiopaque agent; a peptide, a protein, an enzyme, an extracellular matrix component, a cellular component or another biologic agent; captopril, enalapril or another angiotensin converting enzyme (ACE) inhibitor; ascorbic acid, alpha tocopherol, superoxide dismutase, deferoxamine, a 21-amino steroid (lasaroid) or another free radical scavenger, iron chelator or antioxidant; a 14C-, 3H-, 131I-, 32P- or 36S-radiolabelled form or other radiolabelled form of any of the foregoing; estrogen or another sex hormone; AZT or other antipolymerases; acyclovir, famciclovir, rimantadine hydrochloride, ganciclovir sodium or other antiviral agents; 5-aminolevulinic acid, meta-tetrahydroxyphenylchlorin, hexadecaflouoro zinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine betahydroxylase conjugated to saporin or other antibody target therapy agents; enalapril or other prodrugs; any endothelium progenitor cell attracting, binding and/or differentiating agents, including suitable chemoattractive agents and suitable polyclonal and monoclonal antibodies; cell migration inhibiting agents, such as smooth muscle cell migration inhibitors, such as Bamimistat, prolylhydrolase inhibitors, Probacol, c-proteinase inhibitors, halofuginone, and other suitable migration inhibitors; and gene therapy agents, and a mixture of any of these.

Two or more bioactives can be used when preparing a solution for use in a method. For example, FOLFIRI, folinic acid with fluorouracil and irinotecan, is suitable for use as the bioactive in the methods and medical devices described herein. Also, FOLFOX, folinic acid with fluorouracil and oxaliplatin, is suitable for use as the bioactive in the methods and medical devices described herein.

While various example medical devices and methods are described with reference to specific features of particular drawings, it is understood that the various elements, steps and/or features described herein in connection with one particular example can be combined with those of another without departing from the scope of the invention. Furthermore, the medical devices and methods described and illustrated herein provide examples of the invention, and are not intended to limit the scope of the invention in any manner. Rather, they serve only to aid those skilled in the art to perform, make and use the invention.

We claim:

1. A medical device for delivering a bioactive to a point of treatment and retaining the bioactive at the point of treatment for a desired period of time, said medical device comprising:
an elongate shaft extending between proximal and distal ends and having a distal portion extending proximally from the distal end and defining a pigtail;
a balloon disposed on the elongate shaft proximal to the pigtail, the balloon defining an interior chamber;
the elongate shaft defining an inflation lumen extending between an inflation lumen proximal opening and an inflation lumen distal opening disposed within the interior chamber of the balloon;
the elongate shaft defining a first working lumen extending between a first working lumen proximal opening and a first working lumen distal opening disposed between the balloon and the distal end; and
the elongate shaft defining a second working lumen extending between a second working lumen proximal opening and a second working lumen distal opening disposed distal to the first working lumen distal opening and distal to the balloon.

2. The medical device of claim 1, wherein the pigtail comprises a loop.

3. The medical device of claim 2, wherein the pigtail comprises only a single loop.

4. The medical device of claim 3, wherein the pigtail is disposed proximal to the distal end of the elongate shaft such that the distal end is not positioned within the pigtail.

5. The medical device of claim 1, wherein the pigtail comprises a coil.

6. The medical device of claim 1, wherein the first working lumen distal opening is positioned closer to the balloon than to the pigtail on the elongate shaft.

7. The medical device of claim 1, wherein the second working lumen distal opening is disposed on the pigtail.

8. The medical device of claim 7, wherein the second working lumen distal opening is positioned at a point on the pigtail that is opposite a crossover point defined by the elongate shaft in the pigtail.

9. The medical device of claim 1, wherein the second working lumen distal opening is positioned proximal to the pigtail.

10. The medical device of claim 1, wherein the second working lumen distal opening is positioned distal to the pigtail.

11. The medical device of claim 1, wherein the second working lumen distal opening is positioned on the distal end of the elongate shaft.

12. A medical device for delivering a bioactive to a point of treatment and retaining the bioactive at the point of treatment for a desired period of time, said medical device comprising:
an elongate shaft extending between proximal and distal ends and having a distal portion extending proximally from the distal end and defining a pigtail;
a balloon disposed on the elongate shaft proximal to the pigtail, the balloon defining an interior chamber;
the elongate shaft defining an inflation lumen extending between an inflation lumen proximal opening and an inflation lumen distal opening disposed within the interior chamber of the balloon;
the elongate shaft defining a first working lumen extending between a first working lumen proximal opening and a first working lumen distal opening disposed between the balloon and the distal end;
the elongate shaft defining a second working lumen extending between a second working lumen proximal opening and a second working lumen distal opening disposed distal to the first working lumen distal opening and distal to the balloon; and
the elongate shaft defining a second working lumen intermediate opening disposed between the first working lumen distal opening and the second working lumen distal opening, the second working lumen intermediate opening providing fluid access to the second working lumen.

13. The medical device of claim 12, wherein the second working lumen intermediate opening has a size that is the same as a size of the second working lumen distal opening.

14. The medical device of claim 12, wherein the second working lumen intermediate opening has a size that is different from a size of the second working lumen distal opening.

15. The medical device of claim 12, wherein the second working lumen distal opening is positioned on the pigtail.

16. The medical device of claim 15, wherein the second working lumen intermediate opening is positioned proximal to the pigtail.

17. The medical device of claim 12, wherein the second working lumen distal opening is positioned proximal to the pigtail.

18. The medical device of claim 12, wherein the second working lumen distal opening is positioned distal to the pigtail.

19. A medical device for delivering a bioactive to a point of treatment and retaining the bioactive at the point of treatment for a desired period of time, said medical device comprising:
an elongate shaft extending between proximal and distal ends and having a distal portion extending proximally from the distal end and defining a pigtail;
a balloon disposed on the elongate shaft proximal to the pigtail, the balloon defining an interior chamber;
the elongate shaft defining an inflation lumen extending between an inflation lumen proximal opening and an inflation lumen distal opening disposed within the interior chamber of the balloon;
the elongate shaft defining a first working lumen extending between a first working lumen proximal opening and a first working lumen distal opening disposed between the balloon and the distal end;
the elongate shaft defining a second working lumen extending between a second working lumen proximal opening and a second working lumen distal opening disposed distal to the first working lumen distal opening and distal to the balloon; and
the elongate shaft defining a series of second working lumen intermediate openings disposed between the first working lumen distal opening and the second working lumen distal opening, each second working lumen intermediate opening of the series of second working lumen intermediate openings providing fluid access to the second working lumen.

20. The medical device of claim 19, wherein two or more second working lumen intermediate openings are disposed on the pigtail and at least one second working lumen intermediate opening is disposed on the elongate shaft proximal to the pigtail.
